(19) United States
(12) Patent Application Publication
(10) Pub. No.: US 2021/0393057 A1
(43) Pub. Date: Dec. 23, 2021

(54) MULTIFUNCTIONAL PILLOW WITH ADJUSTABLE HEIGHT

(71) Applicant: Zhejiang xieshenghe household textiles Co., Ltd., Hangzhou (CN)

(72) Inventor: Zhixiang YIN, Hangzhou (CN)

(21) Appl. No.: 17/026,215

(22) Filed: Sep. 19, 2020

Related U.S. Application Data
(63) Continuation of application No. PCT/CN2020/100885, filed on Jul. 8, 2020.

(30) Foreign Application Priority Data
Jun. 23, 2020 (CN) 202010579964.1

(51) Int. Cl. A47G 9/10; A47G 9/02; A61B 5/00; A61B 5/0205

(52) U.S. Cl. CPC A47G 9/1027; A47G 9/0253; A61B 5/6892; A61B 7/003; A61B 2562/0247; A61B 2562/066; A61B 5/0205

(57) ABSTRACT

A multifunctional pillow with an adjustable height includes a pillow body and a pillowcase. The fabric shell is stretchable along a length and a height of the fabric shell, but is not stretchable along a width of the fabric shell. The pillowcase is provided with a main space and a plurality of compression spaces, and the different spaces are separated from and communicate with each other by a zipper mechanism, and a mounting pocket is provided at the bottom of the pillowcase. The height of the pillow can be quickly adjusted through the compression spaces. When users sleep on their backs or on their sides, the cervical spine can be supported by adjusting the height of the pillow. The mounting pocket of the pillow is equipped with inflatable cushions and sensors.

### CROSS-REFERENCE TO RELATED APPLICATIONS

This application is a continuation of International Patent Application No. PCT/CN2020/100885 with a filling date of Jul. 8, 2020, which claims the benefit of priority from Chinese Patent Application No. 202010579964.1 with a filing date of Jun. 23, 2020. The content of the aforementioned applications, including any intervening amendments thereto, is incorporated herein by reference.

### TECHNICAL FIELD

The present application relates to beddings, and more particularly to a multifunctional pillow with an adjustable height.

### BACKGROUND

As a bedding item, pillows are capable of supporting necks to allow the neck to have a normal physiology curvature during sleep, so as to maintain normal physiological activities when a person is asleep. Since the designed shape of the pillow is based on the physiological curvature of human bodies, when people sleep on their backs or on their sides, a smooth breathing can be ensured by adjusting heights of pillows. When people lie on the back, the highest point of the pillow should correspond to the middle of the back of the neck to support the cervical flexure, so as to maintain the physiological curvature of the neck. When people lie on the side, the pillow supports the side of the neck, the side of the human body is ensured in an S-shape, and the spine is kept in a straight line.

The existing pillows generally adopt different shapes, and pillows of different shapes have different ergonomic effects. However, such pillow has a single function and cannot be widely applied in modern society. In this case, different pillows should be adopted according to different people and different application scenarios, which is troublesome and causes added cost.

At present, a height-adjustable pillow has been provided in the prior art, in which a pillow body is filled, and block sponges are taken as filling materials of the pillow body. When the height of the pillow is required to be adjusted during use, there is no need to buy extra pillows of different heights. Instead, the height of the pillow can be adjusted by adding or reducing block sponges to the pillow body. However, it is troublesome to clean such pillow, so bacteria are easy to grow in such pillow during the long term use, which will harm human health.

Another height-adjustable pillow is provided, in which the pillow is formed by stacking multiple pillow body sheets and stuffing these pillow body sheets into a pillowcase. The height of the pillow can be adjusted by increasing or decreasing the pillow body sheets. In addition, the pillow body sheet is convenient to clean. However, it is difficult to put these pillow body sheets into the pillowcase simultaneously while keeping them flat, which brings inconvenience to adjust the height of the pillow.

The development of science and technology brings people's increasing demand for pillows. The pillows not only protect the cervical spine during sleep, but also satisfy the need for sleep quality of different application scenarios and different people. For example, people who have poor sleep quality for a long time need to improve sleep quality; people who breathe poorly need to adjust their sleeping posture in order to control their breathing rhythm. In addition, the existing pillows cannot provide proper support angles for those who suffer from cervical spine strain due to long-term work. The elasticity and softness of the existing pillows cannot be adjusted when people change their postures, so the pillow fails to provide sufficient support for the cervical spine, causing damage to the cervical spine.

### SUMMARY OF THE DISCLOSURE

The present disclosure aims to provide a multifunctional pillow with an adjustable height, which is convenient to adjust the height of the pillow to meet the needs of different people in different application scenarios and improve the sleep quality for people who have difficulty in sleep and breathing.

The present disclosure provides a multifunctional pillow with an adjustable height comprises a pillowcase and a pillow body enclosed in the pillowcase; wherein the pillow body comprises a fabric shell and a filling. The fabric shell is stretchable along a length and a height of the fabric shell, and is not stretchable along a width of the fabric shell. A main space and a plurality of compression spaces are located in the pillowcase, and any two of the main space and the plurality of compression spaces are separated from and communicate with each other by a zipper mechanism, and a mounting pocket is provided at a bottom of the pillowcase.

The fabric shell is stretchable along a length and a height of the fabric shell, and is not stretchable along a width of the fabric shell, so that the length and the height of the fabric shell can be adjusted, and the width of the fabric shell is not adjustable.

The pillow body is placed in both the main space and the compression space of the pillowcase, and the main space and the compression space are separated from and communicated with each other by the zipper mechanism. When the zipper mechanism is unfastened, the main space communicates with the compression space. Otherwise, the main space and the compression space are in a separated state.

When people need to increase the height of the pillow in use, the pillow body is squeezed from the compression spaces into the main space. After the pillow body is squeezed out of the compression spaces, the zipper between the main space and the compression spaces is closed, so that the pillow body is completely compressed in the main space. The pillow body is not stretchable along the width of the pillow body. Thus, the compression of the pillow body in a length direction of the pillow body allows the pillow body to extend in a height of the pillow body, so that the height of the pillow body is increased.

When people need to reduce the height of the pillow in use, the zipper between the main space and the compression space is unfastened to allow the main space to communicate with the compression space, so that the pillow body is released from the main space to the compression space. Because the pillow body is stretched in the length of the pillow body, and the pillow body is not stretchable in the width of the pillow body, the height of the pillow body is reduced.

Further, the pillow body is placed in the main space. The compression spaces comprise a first compression space and a second compression space. The first compression space is separated from and communicates with the second compression space and the main space via the zipper mechanism, or the second compression space is separated from and communicates with the first compression space and the main space via the zipper mechanism.

The pillow body is placed in the main space, and the first compression space and the second compression space are configured to adjust the height of the pillow body. The pillow body has a smallest size and a highest height when it is completely located in the main space. The pillow body has a medium size and a medium height when it is filled in the main space and the second compression space. The pillow body has a largest size and a lowest height when it is filled in the main space, the first compression space and the second compression space.

Further, the first compression space and the second compression space are located at ends of the main space. The main space and the first compression space both have a cuboid-like structure. One end of the first compression space is separated from and communicates with the main space through the zipper mechanism, and the other end of the first compression space is separated from and communicated with an outside through the zipper mechanism. The second compression space is located the other end of the main space. One end of the second compression space is separated from and communicates with the main space through the zipper mechanism, and the other end of the second compression space is separated from and communicated with the outside through the zipper mechanism. The zipper mechanism which separates the first compression space from the outside is connected to the zipper mechanism which separates the second compression space from the outside.

The first compression space and the second compression space are located on both sides of the main space, which makes it easy to adjust the height of the pillow. The zipper mechanism which separates the first compression space from the outside and the zipper mechanism which separates the second compression space from the outside can be connected to each other, so as to connect two pillows to form a double pillow, or to connect two ends of one pillow to form a hollow cushion.

Further, the first compression space and the second compression space are located on one end of the main space which has a cuboid-like structure. The second compression space is located at one end of the main space, and the first compression space is located at one end of the second compression space. One end of the second compression space is separated from and communicates with the main space through the zipper mechanism; one end of the first compression space is separated from and communicated with the outside through the zipper mechanism; and the second compression space and the first compression space are separated from and communicated with each other through the zipper mechanism.

The first compression space and the second compression space are located on one end of the main space, so as to adjust the height of the pillow body through directly compressing the first compression space and the second compression space at the same time, thereby realizing the adjustment of the height of the pillow.

In some embodiments, the main space has an irregular hexagonal shape, and the pillow body is placed in the main space. The compression spaces each have a triangular shape, and comprise a first compression space, a second compression space, a third compression space and a fourth compression space which are respectively located at four corners of the pillowcase, and are separated from and communicate with the main space through the zipper mechanism. An opening is respectively arranged at two ends of the pillowcase for connecting an interior of the pillowcase with the outside.

The pillowcase comprises the main space, the first compression space, the second compression space, the third compression space and the fourth compression space. The first, second, third and four compression spaces are respectively located at the four corners of the pillowcase, so as to adjust inclination angles of the pillow. When the pillow body is compressed from the first compression space and the second compression space into the main space, a front of the pillow is lower than a rear of pillow, so that it offers significant support for neck when people sleep on their backs. When the pillow body is compressed from the second compression space and the third compression space into the main space, a left of the pillow is lower than a right of the pillow, so as to offer a significant support for neck when people sleep on the side.

In some embodiments, the mounting pocket is located at the bottom of the pillowcase and has a rectangular shape. A flexible fabric is arranged on the bottom of the mounting pocket, wherein the bottom of the pillowcase and the flexible fabric are connected in a stitched manner. The flexible fabric of the mounting pocket is equipped with a fixing rope. The mounting pocket at a side thereof has an opening.

Further, an inflatable cushion made of rubber is placed in the mounting pocket; the inflatable cushion has a rectangular shape, and comprises an upper air cushion, a lower air cushion and a plurality of supporting air columns. The upper air cushion and the lower air cushion have a cuboid-like structure, and the supporting air columns are cylindrical. The upper air cushion, the lower air cushion and the supporting air columns are communicated with each other, and the supporting air columns are evenly distributed between the upper air cushion and the lower air cushion. The lower air cushion is provided with a deflation valve, and the upper air cushion is provided with an inflation valve.

The inflatable cushion is inflated by the inflation valve. When the inflatable cushion is inflated, the elastic mounting pocket expands, and the volume and height of the inflatable cushion increase. Since the inflatable cushion is located at the bottom of the pillowcase, the height of the pillow is increased due to the increasing height of the inflatable cushion. The inflatable cushion is deflated by the deflation valve. When the inflatable cushion is deflated, the volume and height of the inflatable cushion decrease to reduce the height of the pillow. Therefore, the inflatable cushion can quickly increase or reduce the height of the pillow. The supporting air columns are placed between the upper air cushion and the lower air cushion, which can independently support the user's head and neck to ensure the alignment of the spine and improve the comfort of the user.

In some embodiments, a plurality of sensors are provided in the mounting pocket, and a mounting ring is arranged at the sensors. The mounting ring is detachably connected to the fixing rope on the flexible fabric of the mounting pocket. The sensors comprise a heartbeat sensor, a sound sensor, an air pressure sensor, and a pressure sensor. The heartbeat sensor is placed at a bottom left corner of the mounting pocket, and monitors heartbeats by detecting arteries in a neck of a human body. The sound sensor is placed on a top left corner of the mounting pocket, and detects human breathing sounds. The air pressure sensor is placed on a top right corner of the mounting pocket, and detects air pressure of the inflatable cushion, wherein the air pressure sensor is connected to the deflation valve of the inflatable cushion. The pressure sensor is placed at a bottom right corner of the mounting pocket, and detects a pressure on the pillow. The four sensors communicate with the processor via Bluetooth.

The sensors receive different signals, and the inflatable cushion is automatically inflated according to the different signals. The heartbeat sensor is placed on the bottom left corner of the mounting pocket which is close to the human neck. The heartbeat sensor transmits the data to the processor when it detects fast heartbeats, and then the processor controls the Bluetooth-enabled speaker to play music that helps sleep. The sound sensor, the air pressure sensor and the pressure sensor cooperate with each other. The sound sensor detects the snores and measures the decibels of the snores. The pressure sensor detects the position of the user's head by detecting the pressure point. The inflatable device in the pillow automatically inflates the inflatable cushion according to the air pressure sensor to adjust the height of the pillow, allowing the user to sleep in a comfortable gesture.

In some embodiments, the fabric shell is made of a composite material comprising a polyester material that is not stretchable and elastic cotton that is stretchable, and the fabric shell is made by stitching the polyester material and the elastic cotton.

The fabric shell is made of stretchable high-elastic cotton and non-stretchable polyester material. The fabric shell is stretchable along the length of the fabric shell, but is not stretchable along the width of the fabric shell.

In some embodiments, an opening is respectively provided at the two ends of the pillowcase for connecting the interior of the pillowcase with an outside. The two ends of the pillowcase are separated from and communicate with the outside through a hook and loop fastener or buttons or a zipper.

The hook and loop fastener comprises a first component and the second component which are respectively fixed on the upper surface and the lower surface of the pillowcase. The side of the pillowcase and the outside are separated from and communicated with each other by attaching and tearing apart the first component and the second component of the hook and loop fastener. The buttons are located on the lower surface of the pillowcase, and the upper surface of the pillowcase is provided with buttonholes. The buttons comprise a first button, a second button and a third button, and the buttons are respectively engaged or disengaged with each other. The zipper mechanism includes a belt and a slider. The belt comprises a first teeth row and a second teeth row. The first teeth row is located on the upper surface of the pillowcase, and the second teeth row is located on the lower surface of the pillowcase. The slider moves leftward and rightward to allow the first teeth row and the second teeth row to separate from and communicate with each other.

The present invention has the following beneficial effects. The height of the pillow can be quickly adjusted through the compression spaces. When the users sleep on their backs or on their sides, the cervical spine can be supported by adjusting the height of the pillow. The mounting pocket of the pillow is equipped with inflatable cushions and sensors. According to the data collected by the sensors, the inflatable cushion can quickly adjust the height of the pillow and improve the sleep quality of the users. The fabric shell is made by stitching stretchable elastic cotton and non-stretchable polyester material, so as to adjust the height of the pillow body.

### BRIEF DESCRIPTION OF THE DRAWINGS

FIG. 1 is a side view of a multifunctional pillow with an adjustable height according to an embodiment of the present disclosure.

FIG. 2 is a schematic diagram of the multifunctional pillow according to an embodiment of the present disclosure.

FIG. 3 is a schematic diagram of the multifunctional pillow according to an embodiment of the present disclosure, in which compression spaces are arranged at an end of the multifunctional pillow.

FIG. 4 is a schematic diagram of the multifunctional pillow according to an embodiment of the present disclosure, in which compression spaces are respectively arranged at two ends of the multifunctional pillow.

FIG. 5 is a schematic diagram of the multifunctional pillow according to an embodiment of the present disclosure, in which compression spaces are respectively arranged at four corners of the multifunctional pillow.

FIG. 6 is a schematic diagram of the multifunctional pillow with a mounting pocket according to an embodiment of the present disclosure.

FIG. 7 is a schematic diagram of the multifunctional pillow according to an embodiment of the present disclosure, on which sensors are arranged.

FIG. 8 is a schematic diagram of an inflatable cushion according to an embodiment of the present disclosure.

FIG. 9 is a schematic diagram of a pillow body according to an embodiment of the present disclosure.

In this drawings: pillowcase 1, pillow body 2, main space 3, compression space 4, zipper mechanism 5, mounting pocket 6, first compression space 7, second compression space 8, third compression space 9, fourth compression space 10, air cushion 11, upper air cushion 12, lower air cushion 13, supporting air column 14, heartbeat sensor 15, sound sensor 16, air pressure sensor 17, pressure sensor 18.

### DETAILED DESCRIPTION OF EMBODIMENTS

In view of the shortcomings of the prior art, the present disclosure aims to provide a multifunctional pillow with an adjustable height, which facilitates the adjustment for the height of the pillow to meet the needs of different people in different application scenarios and is able to improve the sleep quality of people who have poor sleep qualities and breathing problems.

The present disclosure adopts the following technical solutions to solve the above technical problems.

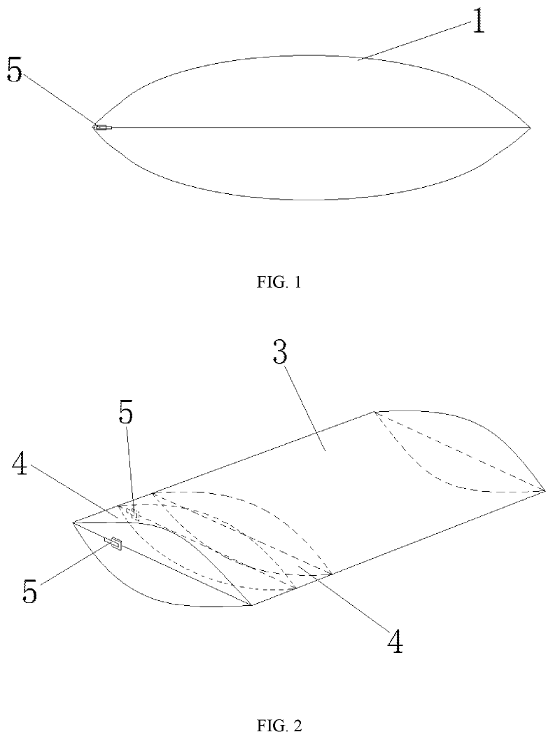

As shown in FIG. 2, this embodiment illustrates a multifunctional pillow with an adjustable height, including a pillowcase 1 and a pillow body 2 in the pillowcase. The pillow body 2 includes a fabric shell and a filling. The fabric shell is stretchable in a length and a height of the pillow body, and is not stretchable in a width of the pillow body. The pillowcase 1 is provided with a main space 3 and a plurality of compression spaces 4, and different spaces are separated and combined by a zipper mechanism 5, and a bottom of the pillowcase 1 is provided with a mounting pocket 6.

Specifically, in some embodiments, the fabric shell is stretchable along a length and a height of the fabric shell, and is not stretchable along a width of the fabric shell, so that the length and the height of the fabric shell can be adjusted, and the width of the fabric shell is not adjustable.

In some embodiments, the pillow body 2 is placed in both the main space 3 and the compression space 4 of the pillowcase 1, and the main space 3 and the compression space 4 are separated from and communicated with each other by a zipper mechanism 5. When the zipper mechanism is unfastened, the main space 3 communicates with the compression space 4. Otherwise, the main space 3 and the compression space 4 are in a separated state.

In some embodiments, when people need to increase the height of the pillow in use, the pillow body 2 is squeezed from the compression space 4 into the main space 3. After the pillow body 2 is squeezed out of the compression space 4, the zipper between the main space 3 and the compression space 4 is closed, so that the pillow body 2 is completely compressed in the main space 3. The pillow body 2 is not stretchable along a width of the pillow body 2. Thus, the compression of the pillow body 2 in a length direction of the pillow body 2 allows the pillow body 2 to extend in a height of the pillow body 2, so that the height of the pillow body 2 is increased.

In some embodiments, when people need to reduce the height of the pillow in use, the zipper between the main space 3 and the compression space 4 is unfastened to allow the main space 3 to communicate with the compression space 4, so that the pillow body 2 is released from the main space 3 to the compression space 4. Because the pillow body 2 is stretched in the length of the pillow body 2, and the pillow body 2 is not stretchable in the width of the pillow body 2, the height of the pillow body 2 is reduced.

In an embodiment, as shown in FIG. 1, the main space 3 located in the pillowcase 1 places the pillow body 2. The compression space 4 includes a first compression space 7 and a second compression space 8 which are separated from and communicate with other spaces through the zipper mechanism 5.

In some embodiments, the main space 3 is configured to place the pillow body 2, and the first compression space 7 and the second compression space 8 are configured to adjust the height of the pillow body 2. The pillow body 2 has a smallest size and a highest height when it is completely located in the main space 3. The pillow body 2 has a medium size and a medium height when it is filled in the main space 3 and the second compression space 8. The pillow body 2 has a largest size and a lowest height when it is filled in the main space 3, the first compression space 7 and the second compression space 8.

In an embodiment, as shown in FIG. 4, the first compression space 7 and the second compression space 8 are located on both ends of the main space 3. The main space 3 and the first compression space 7 both have a cuboid-like structure. One end of the first compression space 7 is separated from and communicates with the main space 3 through the zipper mechanism 5, and the other end of the first compression space 7 is separated from and communicated with the outside through the zipper mechanism 5. The second compression space 8 is located at the other end of the main space 3. One end of the second compression space 8 is separated from and communicates with the main space 3 through the zipper mechanism 5, and the other end of the second compression space 8 is separated from and communicated with the outside through the zipper mechanism 5. The zipper mechanism 5 which separates the first compression space 7 from the outside is connected with the zipper mechanism 5 which separates the second compression space 8 from the outside.

In some embodiments, the first compression space 7 and the second compression space 8 are located on both sides of the main space 3, which makes it easy to adjust the height of the pillow. The zipper mechanism 5 which separates the first compression space 7 from the outside and the zipper mechanism 5 which separates the second compression space 8 of another pillow from the outside can be connected to each other, so as to connect two pillows to form a double pillow, or to connect two ends of one pillow to form a hollow cushion.

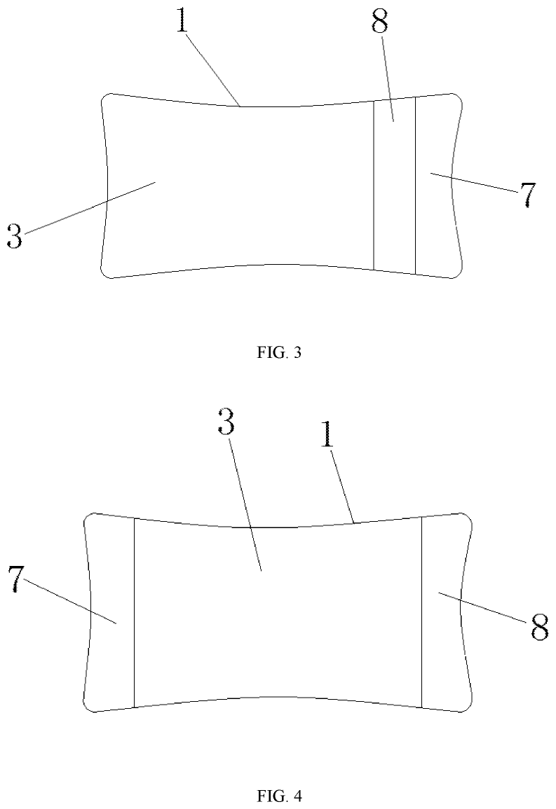

In an embodiment, as shown in FIG. 3, the first compression space 7 and the second compression space 8 are located on one side of the main space 3 which has a cuboid-like structure. The second compression space 8 is located at one side of the main space 3, and the first compression space 7 is located at one side of the second compression space 8. One side of the second compression space 8 is separated from and communicates with the main space 3 through the zipper mechanism 5; one side of the first compression space 7 is separated from and communicated with the outside through the zipper mechanism 5; and the second compression space 8 and the first compression space 7 are separated from and communicated with each other through the zipper mechanism 5.

In some embodiments, the first compression space 7 and the second compression space 8 are located on one side of the main space 3, so as to adjust the height of the pillow body through directly compressing the first compression space 7 and the second compression space 8 at the same time, thereby realizing the adjustment of the height of the pillow.

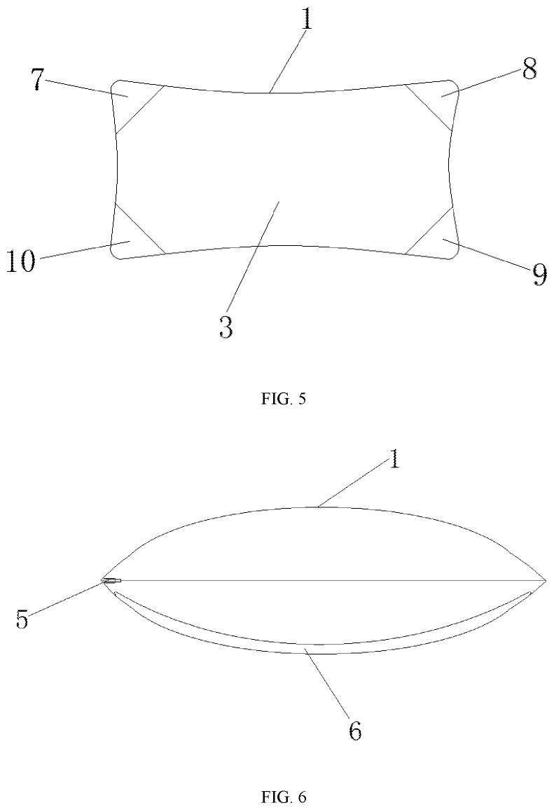

In some embodiments, as shown in FIG. 5, the main space 3 is located in the pillowcase 1, and has an irregular hexagonal shape, and the pillow body 2 is placed in the main space 3. The compression space 4 has a triangular shape, and includes a first compression space 7, a second compression space 8, a third compression space 9 and a fourth compression space 10 which are respectively located at four corners of the pillowcase 1, and separated and combined with the main space 3 through a zipper mechanism 5. A zipper mechanism 5 is respectively arranged at two ends of the pillowcase 1 for connecting an interior of the pillowcase 1 with the outside.

In some embodiments, the pillowcase 1 includes the main space 3, the first compression space 7, the second compression space 8, the third compression space 9 and the fourth compression space 10. The first, second, third and four compression spaces are respectively located at the four corners of the pillowcase 1, so as to adjust inclination angles of the pillow. When the pillow body 2 is compressed from the first compression space 7 and the second compression space 8 into the main space 3, a front of the pillow is lower than a rear of pillow, so that it offers significant support for neck when people sleep on their backs. When the pillow body 2 is compressed from the second compression space 8 and the third compression space 9 into the main space 3, a left of the pillow is lower than a right of the pillow, so as to offer a significant support for neck when people sleep on the side.

In an embodiment, as shown in FIG. 6, the mounting pocket 6 is located at the bottom of the pillowcase 1 and has a rectangular shape. The mounting pocket 6 is formed by stitching the flexible fabric with the bottom of the pillowcase 1. The flexible fabric is equipped with a fixing rope, and the mounting pocket 6 at a side thereof has an opening. The mounting pocket 6 is provided with inflatable cushions 11 and sensors.

In some embodiments, as shown in FIG. 8, the mounting pocket 6 provides an inflatable cushion 11 which is made of rubber and has a rectangular shape. The inflatable cushion 11 includes an upper air cushion 12, a lower air cushion 13 and a plurality of supporting air columns 14. The upper air cushion 12 and lower air cushions 13 are cuboid, and the supporting air columns 14 are cylindrical. The upper air cushion 12, the lower air cushion and the supporting air columns are communicated with each other, and the supporting air columns 14 are evenly distributed between the upper air cushion 12 and the lower air cushion 13. The lower air cushion 13 is provided with a deflation valve, and the upper air cushion 12 is provided with an inflation valve.

In some embodiments, the inflatable cushion 11 is inflated by the inflation valve. When the inflatable cushion 11 is inflated, the elastic mounting pocket 6 expands, and the volume and height of the inflatable cushion 11 increase. Since the inflatable cushion 11 is located at the bottom of the pillowcase 1, the height of the pillow is increased due to the increasing height of the inflatable cushion 11. The inflatable cushion 11 is deflated by the deflation valve. When the inflatable cushion 11 is deflated, the volume and height of the inflatable cushion 11 decrease to reduce the height of the pillow. Therefore, the inflatable cushion 11 can quickly increase or reduce the height of the pillow. The supporting air column 14 is placed between the upper air cushion 12 and the lower air cushion 13, which can independently support the user's head and neck to ensure the alignment of the spine and improve the comfort of the user.

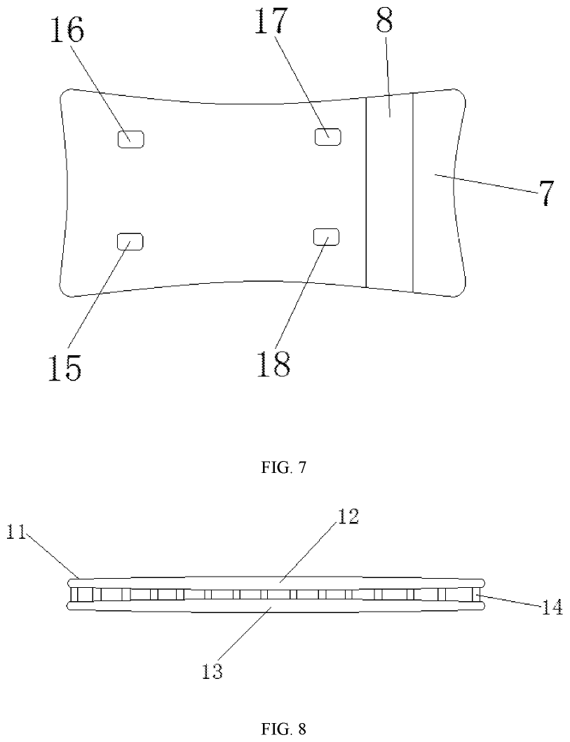

In an embodiment, as shown in FIG. 7, the mounting pocket 6 is provided with sensors having mounting rings. The mounting rings are detachably connected to the fixing rope on the flexible fabric of the mounting pocket 6. The sensors include a heartbeat sensor 15, a sound sensor 16, an air pressure sensor 17, and a pressure sensor 18. The heartbeat sensor 15 is placed on the bottom left corner of the mounting pocket 6, and monitors the heartbeat by detecting the arteries in the neck of the human body. The sound sensor 16 is placed on the top left corner of the mounting pocket 6, and detects human breathing and other sounds. The air pressure sensor 17 is placed on the top right corner of the mounting pocket 6, and detects the air pressure of the inflatable cushion 11. The air pressure sensor 17 is connected to the deflation valve of the inflatable cushion 11. The pressure sensor 18 is placed on the bottom right corner of the mounting pocket 6, and detects the pressure on the pillow. The sensors communicate with a processor via Bluetooth.

In some embodiments, the sensors receive different signals, and the inflatable cushion 11 is automatically inflated according to the different signals. The heartbeat sensor 15 is placed on the bottom left corner of the mounting pocket 6 which is close to the human neck. The heartbeat sensor 15 transmits the data to the processor when it detects fast heartbeats, and then the processor controls the Bluetooth-enabled speaker to play music that helps sleep. The sound sensor 16, the air pressure sensor 17 and the pressure sensor 18 cooperate with each other. The sound sensor 16 detects the snores and measures the decibels of the snores. The pressure sensor 18 detects the position of the user's head by detecting the pressure point. The inflatable device in the pillow automatically inflates the inflatable cushion 11 according to the air pressure sensor 17 to adjust the height of the pillow, allowing the user to sleep in a comfortable gesture.

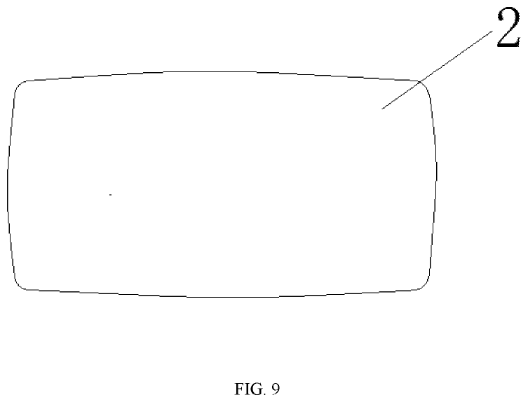

In some embodiments, as shown in FIG. 9, the pillow body 2 has a rectangular structure. The fabric shell is made of a composite material including a polyester material that is not stretchable and high-elastic cotton that is stretchable. The fabric shell is made by stitching the polyester material and the high-elastic cotton.

In some embodiments, the fabric shell is made of stretchable high-elastic cotton and non-stretchable polyester material. The fabric shell is stretchable along the length of the fabric shell, but is not stretchable along the width of the fabric shell.

In some embodiments, two sides of the pillowcase are provided with openings for communicating with the outside, and a side of the pillowcase is separated from and communicates with an outside through a hook and loop fastener or buttons or a zipper.

In some embodiments, the hook and loop fastener includes a first component and the second component which are respectively fixed on the upper surface and the lower surface of the pillowcase. The side of the pillowcase and the outside are separated from and communicated with each other by attaching and tearing apart the first component and the second component of the hook and loop fastener. The buttons are located on the lower surface of the pillowcase, and the upper surface of the pillowcase is provided with buttonholes. The buttonholes include a first buttonhole, a second buttonhole and a third buttonhole. The buttons include a first button, a second button and a third button, and the buttons are respectively engaged or disengaged with each other. The zipper mechanism includes a belt and a slider. The belt includes a first teeth row and a second teeth row. The first teeth row is located on the upper surface of the pillowcase, and the second teeth row is located on the lower surface of the pillowcase. The slider moves leftward and rightward to allow the first teeth row and the second teeth row to separate from and communicate with each other.

The present invention has the following beneficial effects. The height of the pillow can be quickly adjusted through the compression space. When the users sleep on their backs or on their sides, the cervical spine can be supported by adjusting the height of the pillow. The mounting pocket of the pillow is equipped with inflatable cushions and sensors. According to the data collected by the sensors, the inflatable cushion can quickly adjust the height of the pillow and improve the sleep quality of the users. The fabric shell is made by stitching stretchable elastic cotton and non-stretchable polyester material, so as to adjust the height of the pillow body.

Described above are only preferred embodiments of the present disclosure. It should be understood that the above-mentioned embodiments are exemplary, but not intended to limit the scope of the present disclosure. Any change, modification, replacement and variation made by those skilled in the art without departing from the principle and spirit of the present disclosure shall fall within the scope of the appended claims.

What is claimed is:

1. A multifunctional pillow with an adjustable height, comprising:
a pillowcase; and
a pillow body enclosed in the pillowcase;
wherein, the pillow body comprises a fabric shell and a filling; the fabric shell is stretchable along a length and a height of the fabric shell, and is not stretchable along a width of the fabric shell; a main space and a plurality of compression spaces are located in the pillowcase, and any two of the main space and the plurality of compression spaces are separated from and communicate with each other by a zipper mechanism, and a mounting pocket is provided at a bottom of the pillowcase.

2. The multifunctional pillow of claim 1, wherein the pillow body is placed in the main space; the compression spaces comprise a first compression space and a second compression space; the first compression space is separated from or communicates with the second compression space and the main space via the zipper mechanism, or the second compression space is separated from and communicates with the first compression space and the main space via the zipper mechanism.

3. The multifunctional pillow of claim 2, wherein the first compression space and the second compression space are located at ends of the main space; the main space and the first compression space both have a cuboid-like structure; an end of the first compression space is separated from and communicates with the main space through the zipper mechanism, and the other end of the first compression space is separated from and communicated with an outside through the zipper mechanism; the second compression space is located the other end of the main space; an end of the second compression space is separated from and communicates with the main space through the zipper mechanism, and the other end of the second compression space is separated from and communicated with the outside through the zipper mechanism; the zipper mechanism which separates the first compression space from the outside is connected to the zipper mechanism which separates the second compression space from the outside.

4. The multifunctional pillow of claim 2, wherein the first compression space and the second compression space are located on one end of the main space which has a cuboid-like structure; the second compression space is located at one end of the main space, and the first compression space is located at one end of the second compression space; one end of the second compression space is separated from and communicates with the main space through the zipper mechanism; one end of the first compression space is separated from and communicated with the outside through the zipper mechanism; and the second compression space and the first compression space are separated from and communicated with each other through the zipper mechanism.

5. The multifunctional pillow of claim 1, wherein the main space has an irregular hexagonal shape, and the pillow body is placed in the main space; the compression spaces each have a triangular shape, and comprise a first compression space, a second compression space, a third compression space and a fourth compression space which are respectively located at four corners of the pillowcase, and are separated from and communicate with the main space through a zipper mechanism; and an opening is respectively arranged at two ends of the pillowcase for connecting an interior of the pillowcase with the outside.

6. The multifunctional pillow of claim 1, wherein the mounting pocket is located at the bottom of the pillowcase and has a rectangular shape; a flexible fabric is arranged on the bottom of the mounting pocket, wherein the bottom of the pillowcase and the flexible fabric are connected in a stitched manner; the flexible fabric of the mounting pocket is equipped with a fixing rope; and the mounting pocket at a side thereof has an opening.

7. The multifunctional pillow of claim 1, wherein an inflatable cushion made of rubber is placed in the mounting pocket; the inflatable cushion has a rectangular shape, and comprises an upper air cushion, a lower air cushion and a plurality of supporting air columns; the upper air cushion and the lower air cushion have a cuboid-like structure, and the supporting air columns are cylindrical; the upper air cushion, the lower air cushion and the supporting air columns are communicated with each other, and the supporting air columns are evenly distributed between the upper air cushion and the lower air cushion; the lower air cushion is provided with a deflation valve, and the upper air cushion is provided with an inflation valve.

8. The multifunctional pillow of claim 1, wherein a plurality of sensors are provided in the mounting pocket, and a mounting ring is arranged at the sensors; the mounting ring is detachably connected to the fixing rope on the flexible fabric of the mounting pocket; the sensors comprise a heartbeat sensor, a sound sensor, an air pressure sensor, and a pressure sensor; the heartbeat sensor is placed at a bottom left corner of the mounting pocket, and monitors heartbeats by detecting arteries in a neck of a human body; the sound sensor is placed on a top left corner of the mounting pocket, and detects human breathing sounds; the air pressure sensor is placed on a top right corner of the mounting pocket, and detects air pressure of the inflatable cushion, wherein the air pressure sensor is connected to the deflation valve of the inflatable cushion; the pressure sensor is placed at a bottom right corner of the mounting pocket, and detects a pressure on the pillow.

9. The multifunctional pillow of claim 1, wherein the fabric shell is made of a composite material comprising a polyester material that is not stretchable and elastic cotton that is stretchable; and the fabric shell is made by stitching the polyester material and the elastic cotton.

10. The multifunctional pillow of claim 5, wherein an opening is respectively provided at the two ends of the pillowcase for connecting the interior of the pillowcase with an outside; and the two ends of the pillowcase are respectively separated from and communicate with the outside through a hook and loop fastener or buttons or a zipper.
